(19) United States
(12) Patent Application Publication
(10) Pub. No.: US 2015/0098984 A1
(43) Pub. Date: Apr. 9, 2015

(54) NANOLITHOGRAPHY USING LIGHT SCATTERING FROM PARTICLES AND ITS APPLICATIONS IN CONTROLLED MATERIAL RELEASE

(71) Applicant: North Carolina State University, Raleigh, NC (US)

(72) Inventors: Chih-Hao Chang, Cary, NC (US); Xu A. Zhang, Raleigh, NC (US)

(21) Appl. No.: 14/509,672

(22) Filed: Oct. 8, 2014

Related U.S. Application Data
(60) Provisional application No. 61/888,775, filed on Oct. 9, 2013.

(51) Int. Cl. A61K 9/14; G03F 7/20; G03F 7/30

(52) U.S. Cl. CPC A61K 9/14; G03F 7/30; G03F 7/2002
USPC 424/443; 430/325; 428/402; 427/2.21

(57) ABSTRACT

The present disclosure provides hollow nanostructures, methods of forming thereof, and methods of delivery of further nanomaterials utilizing the hollow nanostructures. The hollow nanostructures can be formed by illuminating particles, such as spherical particles, to create a scattering pattern that can be captured on, for example, a photoresist. Thus formed nanoparticles can have a substantially frusto-conical shape that can be tailored based on a variety of factors, including, for example, particle size, particle shape, light exposure characteristics, and light polarization.

### CROSS-REFERENCE TO RELATED APPLICATIONS

This application claims priority to U.S. provisional application number 61/888,775, filed on Oct. 9, 2013, which is incorporated herein by reference in its entirety and for all purposes.

This invention was made with government support under a NASA Office of the Chief Technologist's Space Technology Research Opportunity—Early Career Faculty Grant NNX12AQ46G awarded by NASA and under Grant EEC-1160483 awarded by the National Science Foundation. The government has certain rights in the invention.

### FIELD OF THE INVENTION

The present invention relates to nanostructures formed by light scattering from particles and materials and methods utilizing such forms of nanostructures. More specifically, one or more particles may be illuminated to create a scattering pattern which can be captured by a photoresist to form the nanostructure.

### BACKGROUND OF THE INVENTION

Nanostructures have been the subject of much research in recent years. For example, high surface area-to-volume ratio of some nanostructures has been studied in energy conversion in solar cells and battery electrodes. See, e.g., Fan, Z.; Razavi, H.; Do, J.; Moriwaki, A.; Ergen, O.; Chueh, Y.-L.; Leu, P. W.; Ho, J. C.; Takahashi,T.; Reichertz, L. A.; et al., Three-Dimensional Nanopillar-Array Photovoltaics on Low-Cost and Flexible Substrates, Nat. Mater. 2009, 8, 648-653; and Zhang, H.; Yu, X.; Braun, P. V., Three-Dimensional Bicontinuous Ultrafast-Charge and -Discharge Bulk Battery Electrodes, Nat. Nanotechnol. 2011, 6, 277-281; each herein incorporated by reference in its entirety. Some nanostructures can be used as photonic and phononic crystals. See, e.g., Lin, S. Y.; Fleming, J. G.; Hetherington, D. L.; Smith, B. K.; Biswas, R.; Ho, K. M.; Sigalas, M. M.; Zubrzycki, W.; Kurtz, S. R.; Bur, J., A Three-Dimensional Photonic Crystal Operating at Infrared Wavelengths, Nature 1998, 394, 251-253; Noda, S.; Tomoda, K.; Yamamoto, N.; Chutinan, A., Full Three-Dimensional Photonic Bandgap Crystals at Near-Infrared Wavelengths, Science 2000, 289, 604-606; Qi, M.; Lidorikis, E.; Rakich, P. T.; Johnson, S. G.; Joannopoulos, J. D.; Ippen, E. P.; Smith, H. I., A Three-Dimensional Optical Photonic Crystal with Designed Point Defects, Nature 2004, 429, 538-542; and Jang, J.-H.; Ullal, C. K.; Gorishnyy, T.; Tsukruk, V. V.; Thomas, E. L., Mechanically Tunable Three-Dimensional Elastomeric Network/Air Structures via Interference Lithography, Nano Lett. 2006, 6, 740-743; each herein incorporated by reference in its entirety. High aspect ratio surface nanostructures can also lead to bioinspired anti-reflection and self-cleaning surfaces. See, e.g., MM, W.-L.; Jiang, B.; Jiang, P., Bioinspired Self-Cleaning Antireflection Coatings, Adv. Mater. 2008, 20, 3914-3918; and Park, K.-C.; Choi, H. J.; Chang, C.-H.; Cohen, R. E.; McKinley, G. H.; Barbastathis, G., Nanotextured Silica Surfaces with Robust Superhydrophobicity and Omnidirectional Broadband Supertransmissivity, ACS Nano 2012, 6, 3789-3799; each herein incorporated by reference in its entirety. In biomedical areas, hollow nanostructures and nanoparticles have been investigated for drug delivery systems due to their unique capability to precisely hold and release drugs. See, e.g., Li, Z.-Z.; Wen, L.-X.; Shao, L.; Chen, J.-F., Fabrication of Porous Hollow Silica Nanoparticles and Their Applications in Drug Release Control, J. Controlled Release 2004, 98, 245-254; Lou, X. W.; Archer, L. A.; Yang, Z., Hollow Micro-/Nanostructures: Synthesis and Applications, Adv. Mater. 2008, 20, 3987-4019;Yavuz, M. S.; Cheng, Y.; Chen, J.; Cobley, C. M.; Zhang, Q.; Rycenga, M.; Xie,J.; Kim, C.; Song, K. H.; Schwartz, A. G.; et al., Gold Nanocages Covered by Smart Polymers for Controlled Release with Near-Infrared Light, Nat. Mater. 2009, 8, 935-939; Moon, G. D.; Choi, S.-W.; Cai, X.; Li, W.; Cho, E. C.; Jeong, U.; Wang, L. V.; Xia, Y., A New Theranostic System Based on Gold Nanocages and Phase-Change Materials with Unique Features for Photoacoustic Imaging and Controlled Release, J. Am. Chem. Soc. 2011, 133, 4762-4765; and Tao, S. L.; Desai, T. A., Microfabricated Drug Delivery Systems: From Particles to Pores, Adv. Drug Delivery Rev. 2003, 55, 315-328; each herein incorporated by reference in its entirety. Various “top-down” 3D lithography approaches have been used to enable these advances, including layer-by-layer techniques, focused ion beam milling, and electron-beam lithography. See, e.g., Jeon, J.; Floresca, H. C.; Kim, M. J., Fabrication of Complex Three-Dimensional Nanostructures Using Focused Ion Beam and Nanomanipulation, J. Vac. Sci. Technol., B 2010, 28, 549-553; and Yamazaki, K.; Yamaguchi, H., Three-Dimensional Alignment with 10 nm Order Accuracy in Electron-Beam Lithography on Rotated Sample for Three-Dimensional Nanofabrication, J. Vac. Sci. Technol., B 2008, 26, 2529-2533; each herein incorporated by reference in its entirety. While these techniques can be used to create nanostructures, they can be costly and difficult to scale up for manufacturing.

There remains a need in the art for further nanostructures with advantageous properties and methods for forming such nanostructures.

### SUMMARY OF THE INVENTION

The present invention provides methods for forming hollow nanostructures comprising the use of particle light scattering nanolithography. In the present methods, particles can be used to focus and scatter light onto a photoresist and thereby form nanostructures exhibiting properties that can be defined by the scattering characteristics. The intensity pattern recorded by the underlying photosensitive materials results in hollow shell-like structures that can provide multiple advantages as discussed herein.

Various embodiments of the methods of making a hollow nanostructure as disclosed herein comprise positioning one or more micro- or nano-sized particles over a photoresist, illuminating the particle, thereby forming a scattering pattern in the photoresist, and developing the photoresist to reveal the hollow nanostructure defined by the scattering pattern. In some embodiment, the particles can have a diameter of about 50 μm or less. In certain embodiments, the particle can have a diameter in the range of about 200 nm to about 2000 nm. In addition, the method can further comprise illuminating the particle with an exposure laser having a wavelength of about 325 nm to about 405 nm. In various embodiments, the particle can have a diameter in the range of about 5 nm to about 50 nm. The method can further comprise illuminating the particle with an exposure having a wavelength of about 1 nm to about 10 nm. Furthermore, in various embodiments of the present invention, the scattering pattern can correspond to a ratio γ of the particle diameter D to the exposure wavelength λ (γ32 D/λ).

In an embodiment, the Mie scattering regime for a spherical particle can be used, wherein the particle diameter D is comparable to wavelength λ. In this regime, the angular-variant scattering profile can result in complex intensity patterns. Masks are not necessary in this approach (although they may be incorporated if desired), and scattering particles can be assembled directly on the photoresist. In particular, colloidal light scattering can be used to fabricate three-dimensional (3D) geometries. In some embodiments, dielectric particles (e.g., spheres) can be used as scattering particles. In other embodiments, metallic nanoparticles can be used in the alternative to enable localized plasmonic interactions for subwavelength patterning to fabricate other nanostructures. For example, such methods can be used for 3D visualization of near-field enhancement of plasmonic nanostructures.

Various embodiments of a nanostructure described herein comprise a base, a top with an opening formed thereon, and a sloped sidewall connecting the base to the top. In certain embodiments, the combination of the base, top, and sidewall forms a frusto-conical shape having an angle θ. In various embodiments, the angle θ can range from about 45 to about 90 degrees. The nanostructure can also comprise a hollow chamber defined by the base and sidewall and communicating with the top opening. In some embodiments, a nanostructure array comprising a plurality of such nanostructures can be provided. The plurality of the nanostructures can be attached to a backing material.

In various embodiments of the present invention, a drug delivery device can comprise an array of nanostructures as described herein. In some embodiments, a method of forming a nanomaterial delivery device comprises providing a hollow nanostructure as described herein and contacting the hollow nanostructure with a composition comprising a nanomaterial and a carrier so that the nanomaterial enters the hollow chamber. Various embodiments of a method for delivering a nanomaterial comprise providing a hollow nanostructure according to the present invention, wherein the hollow nanostructure has the nanomaterial retained within the hollow chamber and then subjecting the hollow nanostructure to conditions such that the nanomaterial is released from the hollow chamber. As a non-limiting example, a phase change material may be included in the hollow chamber to retain the nanomaterial therein, and the phase change material may undergo a change at the desired time and/or location of delivery (e.g., a change from solid to liquid, from liquid to gas, or from solid to gas) to facilitate release of the nanomaterial from the hollow chamber.

### DESCRIPTION OF THE DRAWINGS

Having thus described the present disclosure in general terms, reference will now be made to the accompanying drawings, which are not necessarily drawn to scale, and wherein:

FIG. 1 illustrates a method of fabricating hollow nanostructures according to the present disclosure using light scattering from a colloidal nanoparticle, wherein (a) illustrates finite-difference time-domain (FDTD) simulation showing the scattering intensity pattern produced by transverse electric (TE)-polarized UV light and a single nanosphere, wherein shaded areas indicate high intensities; (b) illustrates recordation of an intensity pattern by an underlying photoresist layer on top of an anti-reflective coating (ARC) layer; and (c) shows a sectioned diagram of a resulting hollow nano-volcano structure;

FIG. 2 (a through 1) illustrates micrographs of fabricated hollow nanostructures according to the present disclosure using TE-polarized and transverse magnetic (TM)-polarized 325 nm illuminations;

FIG. 3 illustrates structure prediction using FDTD and binary resist models for nano-volcanos according to the present disclosure where diagrams (a) and (b) compare the side geometries between fabricated and simulated structures, respectively, while (c) and (d) present a quantitative comparison of the same fabricated and simulated nano-volcano interiors, respectively;

FIG. 4 is a graph illustrating operating shape diagram of the patterned structures according to the present disclosure and shows the experimental results and FDTD simulations of the sidewall angle θ with respect to diameter/wavelength (γ);

FIG. 5 shows micrographs illustrating the polarization effect on the structural symmetry of nanostructures according to the present disclosure where (a) shows a top view of a nanostructure fabricated using a linearly polarized 325 nm laser and 500 nm diameter particles with exposure dose of 130 mJ/cm² overlaid with the simulated structure; and (b-d) show top, side and cross-section views, respectively, of the nanostructures fabricated using an unpolarized mercury lamp narrow-band filtered at 365 and 500 nm diameter particles;

FIG. 6 (a through c) shows micrographs of periodic nano-volcano arrays according to the present disclosure with differing periods and heights; and

FIG. 7 shows micrographs of particle-trapping nano-volcanoes according to the present disclosure where 50 nm diameter silica particles are trapped in (a) double-shelled and (b) thick-walled nano-volcanoes.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

The present invention provides a method for the fabrication of hollow nanostructures using light scattering from one or more particles and its application in trapping of further nanomaterials. This approach harnesses the local optical interaction with particles, (such as colloidal particles, for example) and allows versatile, facile fabrication of nanostructures. While external optics may be used if desired, they are not required in this approach, and light can be manipulated into the designed optical pattern solely by the particles. The scattering field is well defined by the proposed shape diagram, and various geometries can be readily fabricated by controlling the ratio of particle size to light wavelength utilized. The nanostructures can also be patterned with periodic order using non-close-packed particle assembly, allowing precise spatial control for particle trapping/delivery, for example. The nanoscale interior volume of the hollow nanostructures can be effective in trapping nanoparticles and can potentially be used in drug delivery and delivery of other nanomaterials. Further, non-limiting examples of the present nanostructures include biosensing and optical or acoustic focusing using the tapered sidewalls. Although the present disclosure particularly exemplifies spherical nanoparticles as scattering objects, in various embodiments, non-spherical particles, such as cubic, tetrahedral, and rod particles, can be incorporated into this approach to enable a wide variety of nanostructures.

The present invention provides methods for forming hollow nanostructures that can comprise the use of particle light scattering nanolithography. Various embodiments of the present methods of making a hollow nanostructure can comprise positioning one or more particles over a photoresist, illuminating the particles, thereby forming a scattering pattern in the photoresist, and developing the photoresist to reveal the hollow nanostructure defined by the scattering pattern. The nanostructures formed by the present methods can exhibit properties that can be governed by the scattering characteristics.

In some embodiments, the particles can be microparticles and/or nanoparticles. In particular embodiments, particles used according to the disclosure can have an average size of about 50 μm or less, about 10 μm or less, or about 2 μm or less. For example, particles can have an average size of about 1 nm to about 10 μm, about 5 nm to about 5 μm, or about 200 nm to about 2 μm. It should be noted that particle size can depend on the wavelength of the exposure laser. Therefore, other particle sizes correlating to alternative exposure wavelengths are contemplated by the methods described herein. For example, in various embodiments nanoparticles with a diameter ranging from about 200 nm to about 2000 nm can be used for a wavelength of 325 nm. For x-rays or extreme UV (wavelength of 1-10 nm), nanoparticles with a diameter ranging from about 5 nm to about 50 nm can be used, for example.

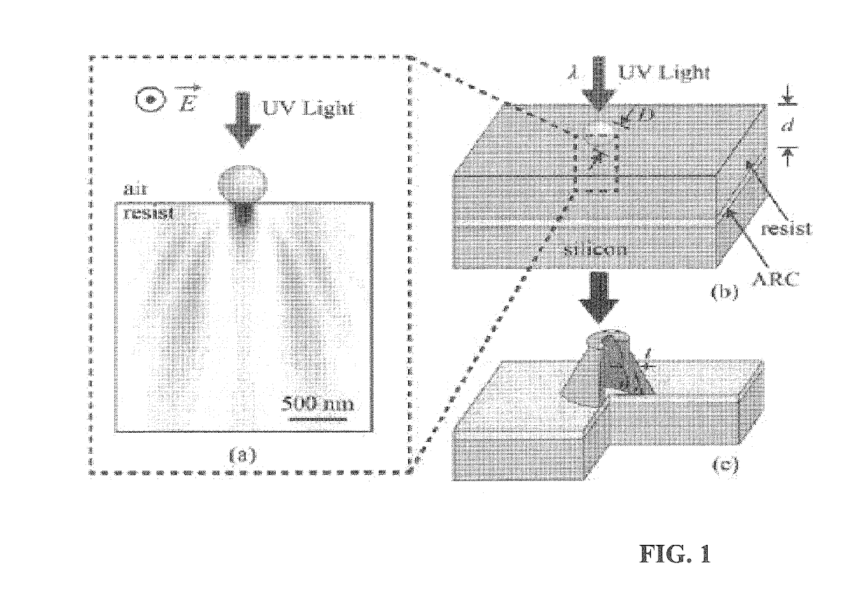

In an embodiment of a method of fabricating a hollow nanostructure, as illustrated in FIG. 1, for example, a single nanosphere can scatter normal-incident UV light into one main and multiple side lobes to form an intensity distribution. The scattering field can be calculated using finite-difference time-domain (FDTD) method, as disclosed, for example, in Oskooi, A. F.; Roundy, D.; Ibanescu, M.; Bermel, P.; Joannopoulos, J. D.; Johnson, S. G., Meep: A Flexible Free-Software Package for Electromagnetic Simulations by the FDTD Method, Comput. Phys. Commun. 2010, 181, 687-702, which is herein incorporated by reference in its entirety. An underlying photoresist layer (or similar material) can be used to record this intensity pattern, as shown in Figure lb, for example. In some embodiments, a positive-tone photoresist is used and a hollow shell-like “nano-volcano” can be obtained, as illustrated in FIG. 1c, for example. These structures can be hollow, with sloped walls and with one opening located on the top side of the structure. The slanted shell can be defined by the minima of the side lobes, and its angle θ and thickness t can be controlled.

An important parameter in the nanostructure formation process is the ratio γ of the particle diameter D to the exposure wavelength λ(γ=D/λ), which can determine the scattering regime. The diameter of the particle used can be selected to be comparable to the exposure wavelength so the light scattering would be in the Mie scattering regime (γ˜1), resulting in alternating bright and dark side lobes that define the nanostructure shells. Outside of this regime, the scattered light can either result in uniform angular distribution, as observed in the Rayleigh scattering (γ<<1), or in mostly focusing, as shown in the solution of geometrical optics (γ>>1).

The wavelength of the exposure laser can depend on the absorption property of the photoresist used. Wavelengths of the exposure laser can range, for example, from about 1 to about 1000 nm. In various embodiments of the present method, a UV exposure laser can be used. For example, an exposure laser with a wavelength of about 325 to about 405 nm can be used. In some embodiments, a smaller wavelength exposure laser can be used. For example, an x-ray or an extreme UV laser with a wavelength of about 1 to about 10 nm can be used. In various embodiments, a visible or near-infrared exposure laser can be used. For such a high-wavelength exposure, the photoresist materials can be less absorbing and can result in taller nanostructures.

Various embodiments of a nanostructure described herein can comprise one or more shells comprising a base, a top with an opening formed thereon, and a sloped sidewall connecting the base to the top and forming a frusto-conical shape having an angle θ. The nanostructure further comprises a hollow chamber defined by the base and sidewall and communicating with the top opening. While the structures described herein can be patterned in polymeric materials, other materials can be introduced by using conformal deposition techniques such as atomic layer deposition. This can allow the formation of structures that are mechanically more stable and can enable further surface functionalization of the nanostructures. Also, by changing the scattering regime used to form the nanostructure, different characteristics can be achieved. In some embodiments, a nanostructure formed according to the present disclosure can be utilized as a template for formation of non-polymeric nanostructures.

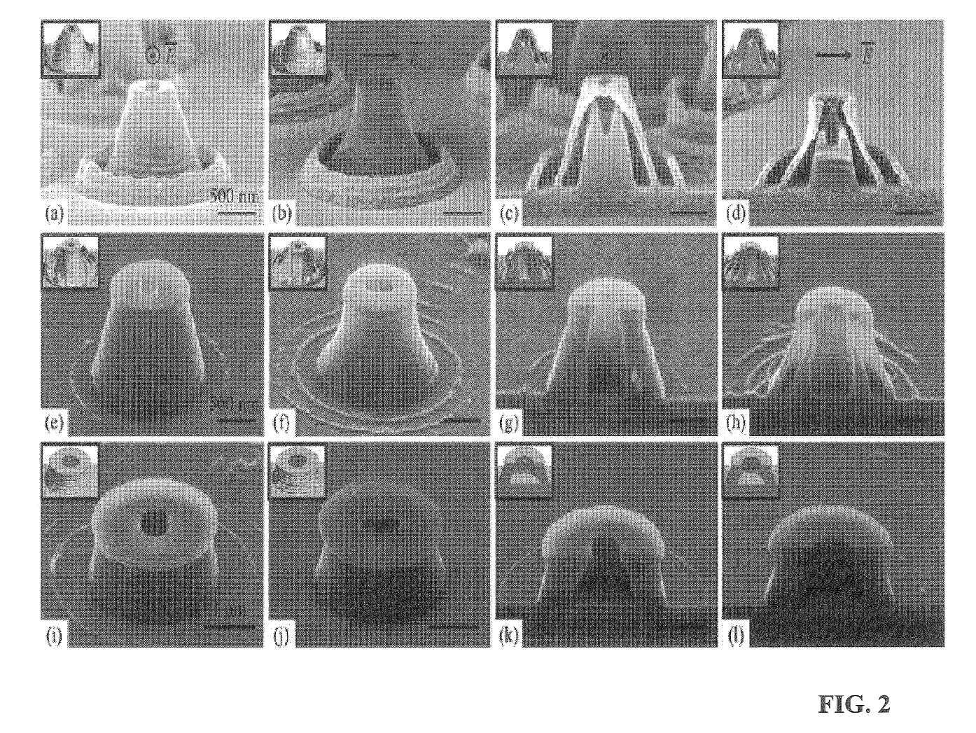

By varying and controlling γ in the Mie scattering regime, patterned nanostructures can be designed. In some embodiments, by varying the particle diameter-to-wavelength ratio γ, the structures can be designed with and without an inner core, as shown, for example, in FIG. 2a-d and FIG. 2e-l, respectively. In addition, the angle of the sidewall can be dependent on γ and can be designed to be a desired angle. For example, in various embodiments the sidewall angle can range from about 45 to about 90 degrees, or about 65 to about 85 degrees. The exposure dose is another factor that can affect sidewall thickness and the inner core geometry. Exposure time and intensity define the exposure dose which has effects on the resulting geometry of the fabricated nanostructure. For example, although sidewall angles of the fabricated nanostructures do not change with the exposure dose, sidewall thickness is affected by the exposure dose. The shape of the top opening of the fabricated nanostructures can also be affected by the exposure dose, for example. The particle light scattering can be described using FDTD and binary resist models developed, and the nanostructure dimensions can be accurately predicted.

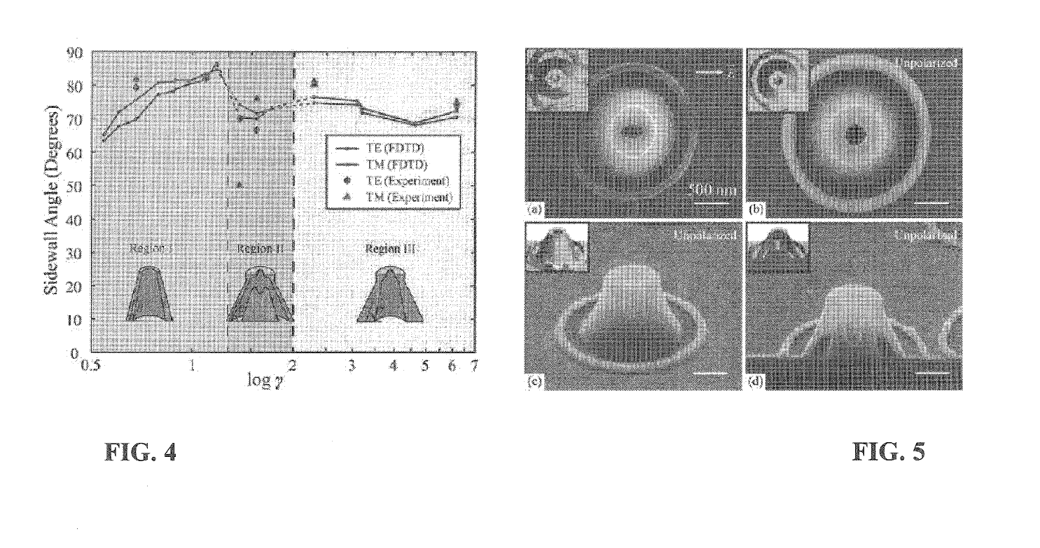

Since the lithographic exposure can be governed by the light scattering profile in the Mie scattering regime, the fabricated structure can be designed by controlling the particle diameter-to-wavelength ratio γ. To illustrate this dependency, the sidewall angles of the nano-volcanoes can be compared with respect to different exposure γ values, as shown in FIG. 4 for example. The sidewall angle values can be experimentally measured using cross-section scanning electron microscopy (SEM) micrographs and compared with theoretical predictions using the FDTD method. Three regions of the achievable hollow nano-volcano structures are presented in FIG. 4. The dashed lines connecting the adjacent regions represent transitional regions, where shell damages may occur. The black dashed lines are representative boundaries between regions, which are given by γ=1.3 and 2 in an embodiment.

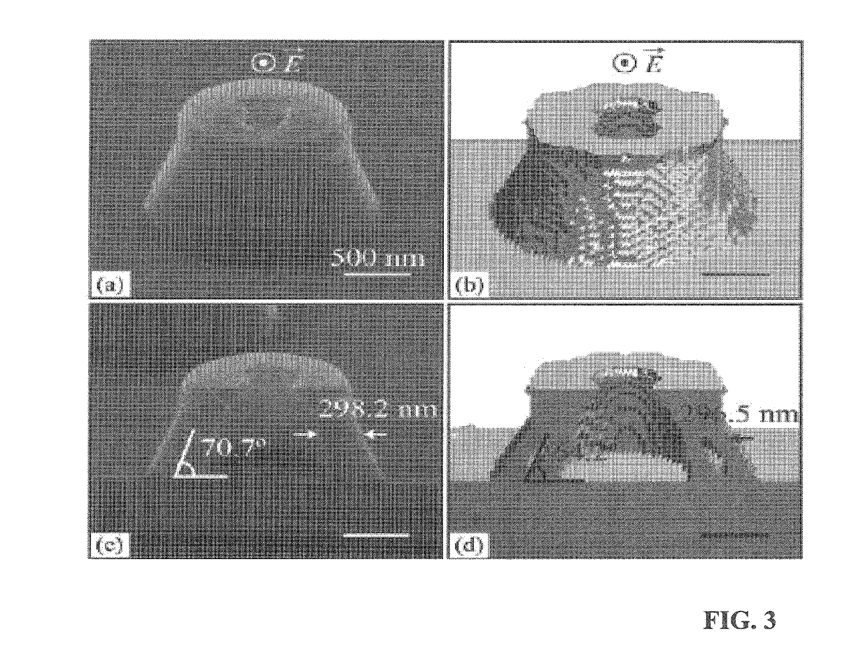

Based on the values of γ, the geometries of the nano-volcanoes can be divided into three regimes, as represented by the three regions illustrated in FIG. 4, for example. In region I, where γ<1.3 in the illustrated embodiment, the intensity minima of the first side lobes of the colloidal scattering pattern define the outer sidewall, and thus the angle increases as γ increases. The structures in this region are coreless and have nearly vertical sidewalls when γ approaches 1.3, which defines the boundary (black dashed line) between regions I and II. The dashed lines connecting regions I and II represent the transitional region (where 1.2<γ<1.4 in the illustrated embodiment) and the nano-volcanoes may collapse due to thin and porous shells. In region II, the intensity minima of the second side lobes define the outer sidewalls, resulting in lower angles than those defined by the first intensity lobes. The first side lobes still exist at higher scattering angles, resulting in the formation of an inner core within the hollow structure, as shown in FIG. 4, for example. Because the sidewalls are usually very thin in this region (˜60-100 nm), the shell can sometimes sag due to mechanical instability, as indicated in the curved sidewall shown in FIG. 2d for example (where γ=1.38 in the illustrated embodiment). Note that the simulated pattern using FDTD for the corresponding exposure condition predicts straight sidewalls. In region III (where γ>2 in the illustrated embodiment) hollow and coreless nanostructures with thick sidewalls can be fabricated. The thick sidewall results from a combination of the first and second side lobes, as the scattering field becomes too close to form separate structures and results in a sidewall with undeveloped volume, as seen in FIG. 3 for example. FIG. 4 gives a comprehensive summary of the three regions, representing an operating shape diagram to the available nanostructures that can be patterned. Based on specific application, the appropriate geometry can be chosen by designing the corresponding γ value. Note that the shape diagram shown in FIG. 4 is unitless and it can be scaled to fabricate nanostructures of any length scale.

In addition to γ and exposure dose, the polarization state of the incident light can also have an impact on the patterned structures. Since the spherical colloidal elements described for exemplary purposes in this application are circularly symmetric, the symmetry of the patterned nano-volcanoes is solely dependent on the polarization state of the illumination. The fabrication results using a linearly polarized laser illustrate the two-fold symmetry of the nanostructures, as shown in FIG. 2, for example. This symmetry can be better observed in the top-view micrograph for a sample with γ=1.54, as shown in FIG. 5a for example, where the long axis of the elliptical opening is aligned to the polarization direction. When the exposure light is unpolarized, the fabricated nanostructures can have cylindrical symmetry, as shown in FIG. 5b-d, for example These exemplary structures were fabricated using 500 nm diameter particles and an unpolarized mercury lamp narrow-band filtered at 365 nm. The value of γ is in region II of FIG. 4, and an inner core can be observed in the nano-volcano. Therefore, the phase diagram illustrated in FIG. 4 can also be applicable to unpolarized illuminations. The shape of the particle can also affect the symmetry of the resultant nanostructures. If non-spherical particles are used, such as cubic particles or nanorods, the resultant structures can resemble the symmetry of the elements and more complex geometries can be obtained.

In some embodiments, a nanostructure array comprising a plurality of nanostructures as described herein can be provided. The plurality of the nanostructures can be attached to a backing material. For example, in an embodiment, the hollow nanostructures can be patterned in a periodic array using light scattering from hexagonal non-close-packed particles, which can allow for precise spatial control of the surface structures. In this approach, individual nanoparticles can be placed in a periodic array that has a larger period than the particle diameters. This particular type of assembly can be achieved, for example, via isotropic oxygen plasma etching of a monolayer of close-packed micrometer-size polystyrene nanospheres. The plasma etching can preserve the spherical shape and the periodic order of the particles while reducing the particle diameter.

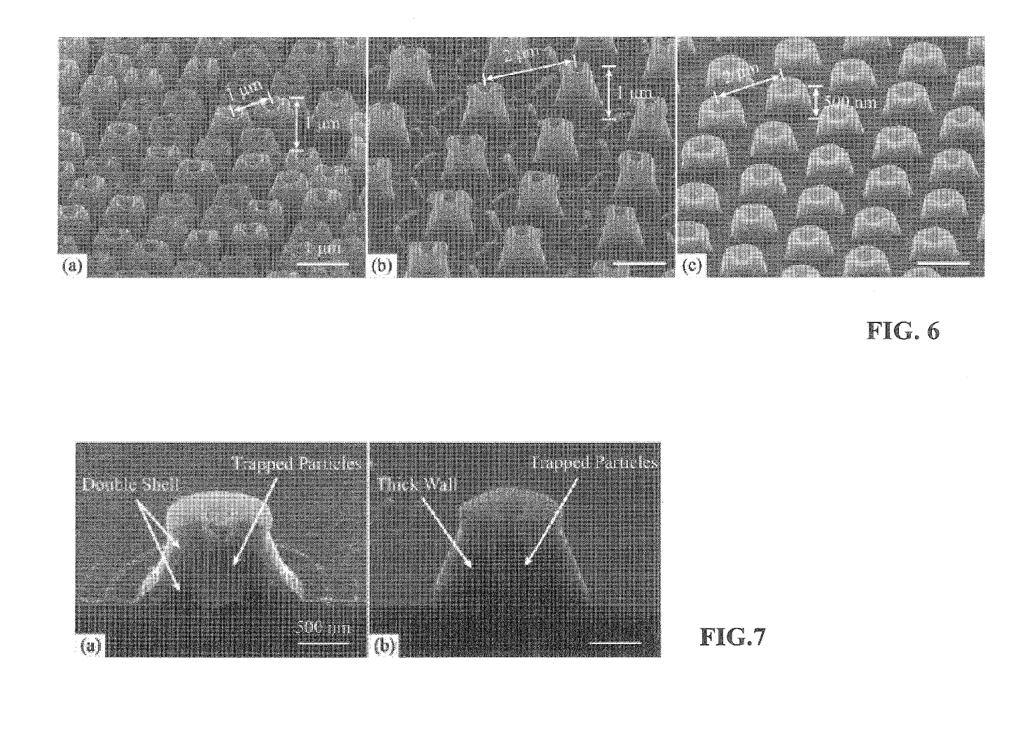

Various types of periodic arrays using different initial sphere diameters and resist thicknesses can be fabricated. For example, FIG. 6 illustrates three different types of periodic arrays with (a) 1 μm period and 1 μm height, (b) 2 μm period and 1 μm height, (c) 2 μm period and 500 nm height. The individual structure in each array has a hollow structure, similar to isolated ones fabricated individually. The periods can depend on the initial size of the nanospheres prior to size reduction and in an embodiment are 1 and 2 μm, as shown in FIG. 6 a and b, respectively. Structural defects, such as broken lower sidewalls exemplified in FIG. 6a can be observed due at least in part to light interference between overlapping scattering fields from neighboring particles. By reducing the thickness of the photoresist to 500 nm, for example, the same 2 μm period array can have improved structural quality, as shown in FIG. 6c. The thickness of photoresist can be chosen within the height limit to avoid light interference between neighboring particles, which can depend on the particle array period and the particle diameter-to-wavelength ratio.

The thickness of the photoresist layer preferably is sufficient to capture the scattering pattern and can relate to the particle size used. Photoresist thickness in some embodiment is limited by the absorption of the photoresist material, as light intensity decays exponentially into the material. In non-limiting examples, a photoresist utilized according to the present disclosure may have a thickness of about 0.1 μm to about 2 μm, about 0.25 μm to about 1.75 μm, or about 0.5 μm to about 1.5 μm.

In various embodiments of periodic arrays, the lower bound of the period can be the same order of magnitude as the diameter of the particle used to fabricate each nanostructure in the array. For example, the lower bound can range from about 1 nm to about 10 μm. The upper bound of the period can be greater depending on the density of nanostructures desired. For example, the period can be about 50 μm to about 100 μm.

Similarly, the height of the nanostructures, either individually or in an array, can correlate to the particle diameter. In various embodiments, the height of a nanostructure can range from about 1 nm to about 10 μm, or about 5 nm to about 5 μm, or about 500 nm to about 1.5 μm. Taller structures can be fabricated from larger diameter particles, for example.

Embodiments of the method described herein can harness particle light scattering for fabrication of hollow nanostructures, which can find applications in functionalized surfaces for particle/cell trapping and drug delivery. In various embodiments of the present invention, a drug delivery device can comprise an array of nanostructures as described herein. Given the nanoscale volume of each nanostructure, a finite number of particles can be trapped, which can be used, for example, for precise drug delivery. Hollow nanostructures have drawn particular interests in these areas. The presently disclosed nanostructures and nanostructure arrays can utilize a wide variety of drug loading and release mechanisms including but not limited to those described in Li, Z.-Z.; Wen, L.-X.; Shao, L.; Chen, J.-F. Fabrication of Porous Hollow Silica Nanoparticles and Their Applications in Drug Release Control. J. Controlled Release 2004, 98, 245-254; Lou, X. W.; Archer, L. A.; Yang, Z. Hollow Micro-/Nanostructures: Synthesis and Applications. Adv. Mater. 2008, 20, 3987-4019; Yavuz, M. S.; Cheng, Y.; Chen, J.; Cobley, C. M.; Zhang, Q.; Rycenga, M.; Xie,J.; Kim, C.; Song, K. H.; Schwartz, A. G.; et al. Gold Nanocages Covered by Smart Polymers for Controlled Release with Near-Infrared Light. Nat. Mater. 2009, 8, 935-939; Moon, G. D.; Choi, S.-W.; Cai, X.; Li, W.; Cho, E. C.; Jeong, U.; Wang, L. V.; Xia, Y. A New Theranostic System Based on Gold Nanocages and Phase-Change Materials with Unique Features for Photoacoustic Imaging and Controlled Release. J. Am. Chem. Soc. 2011, 133, 4762-4765; and Tao, S. L.; Desai, T. A. Microfabricated Drug Delivery Systems: From Particles to Pores. Adv. Drug Delivery Rev. 2003, 55, 315-328; each herein incorporated by reference in its entirety.

In some embodiments, a method of forming a nanomaterial delivery device comprises providing a hollow nanostructure as described herein, and contacting the hollow nanostructure with a composition comprising a nanomaterial and a carrier so that the nanomaterial enters the hollow chamber. For example, methods discussed above for drug loading mechanisms may be utilized in formation of a nanomaterial delivery device according to the present disclosure.

Various embodiments of a method for delivering a nanomaterial comprise providing a hollow nanostructure according to the present invention, wherein the hollow nanostructure has the nanomaterial retained within the hollow chamber. The method further comprises subjecting the hollow nanostructure to conditions such that the nanomaterial is released from the hollow chamber. In an exemplary embodiment, a phase change material may be utilized to retain the nanomaterial within the hollow chamber. To facilitate delivery of the nanomaterial, the phase change material may be adapted to change its state (e.g., from a solid to a liquid, from a liquid to a gas, or from a solid to a gas) at a defined location or under defined conditions. The state change can allow release of the previously retained nanomaterial from the hollow chamber. In some embodiments, focused light and/or focused sound can be used to locally heat a part of an array to selectively release one or more nanostructures, for example. In various embodiments, patterned electrical current can be used to release one or more nanostructures through resistive heating, for example. Further release mechanisms, such as those discussed above for drug release may be utilized in the methods of the present disclosure.

The following examples are provided to illustrate further the present invention, but should not be construed as limiting the scope thereof. Unless otherwise noted, all parts and percentages are by weight.

### p EXPERIMENTAL

The present invention is more fully illustrated by the following examples, which are set forth to illustrate the present invention and are not to be construed as limiting thereof. In the following examples, nm means nanometer and pm means micrometer. All weight percentages are expressed on a dry basis, meaning excluding water content, unless otherwise indicated.

In all experiments, the samples were prepared on silicon substrates. A layer of anti-reflection coating (ARC) (i-CON-16, Brewer Science, Inc.) was used to reduce back reflection. The ARC thickness was around 90 nm and 160 nm for the 325 nm and 365 nm wavelength exposures, respectively. Positive photoresist (Sumitomo PFi88A7) was spin-coated on the ARC layer. To enhance sphere adhesion and facilitate the spreading of the aqueous colloidal solution, a 10 nm thick layer of silicon dioxide was deposited on top of the resist surface using electron-beam evaporation. It is designed to be thin enough to have minimal optical effects on the scattering pattern. Monodisperse polystyrene spheres with various diameters from 350 nm to 1.9 μm (Polyscience Polybead Microspheres in 2.5% aqueous solution) were used as scattering objects in the experiments. The solution was spin-coated on the prepared sample to assemble isolated nanospheres.

Lithographic exposures were performed using either a linearly polarized 325 nm He-Cd laser or an unpolarized mercury lamp narrow-band filtered at 365 nm. After exposing the samples with doses of 100-200 mJ/cm², the nanospheres were removed by ultrasonic agitation and the thin silicon dioxide layer was etched using buffered hydrofluoric acid (HF) (J. T. Baker, buffered oxide etch 10:1). The exposed samples were then developed in 2.4% tetramethylammonium hydroxide (TMAH) developer solution (Micropo sit MF-CD-26) for 1-2 min The samples were characterized using top view and cross-section view scanning electron microscope (JEOL 6400F) at 5 keV.

The colloidal light scattering intensity distribution in the photoresist was modeled using finite-difference time-domain (FDTD) methods. See, e.g., Kühler, P.; García de Abajo, F. J.; Solis, J.; Mosbacher, M.; Leiderer, P.; Afonso, C. N.; Siegel, J., Imprinting the Optical Near Field of Microstructures with Nanometer Resolution, Small 2009, 5, 1825-1829, which describes a two dimensional simulation. The results were also compared with the results from the analytical Mie scattering solutions. A binary photoresist model was used to predict the resulting structures, where any volume above a threshold dose was completely removed. The intensity profile through the thickness of the resist was assumed to have an exponential decay to model the resist absorption. The geometries of the simulated structures are then quantitatively analyzed in Matlab. The predicted models are depicted as inset diagrams with their corresponding fabrication results.

### EXAMPLE 1

Various evaluation methods were used to characterize nanostructures that were prepared as described above. Scanning electron microscope (SEM) images of the fabricated nanostructures are shown in FIG. 2, for example. FIG. 2 illustrates micrographs of the fabricated hollow nanostructures using TE-polarized and TM-polarized 325 nm illuminations. The inset diagrams show the corresponding simulated nanostructures using the FDTD method and a binary resist model. The following conditions were utilized in the formation of the respective nanostructures. For the nanostructures illustrated in FIGS. 2 a-d particle diameter D=450 nm, exposure dose=130 mJ/cm², and γ=1.38. For the nanostructures illustrated in FIGS. 2 e-h D=750 nm, exposure dose=120 mJ/cm², and γ=2.30. For the nanostructures illustrated in FIGS. 2 i-l D=1.9 μm, exposure dose=130 mJ/cm², and γ=5.85.

In these evaluations, linearly polarized 325 nm laser was used to illuminate particles with diameters of 450 nm, 750 nm, and 1.9 μm. The micrographs in each row of FIG. 2 represent the side and cross-section views of the hollow nanostructures patterned with TE-polarized and TM-polarized light, respectively, for the three different diameter spheres. Minor differences between the different polarization exposures can be observed, indicating the patterned nanostructures have a two-fold symmetry. The inset in each micrograph depicts the simulated structure using FDTD method and a binary resist model. Mie theory can also be used to predict the angular intensity profile of the scattered pattern but cannot accurately model the effect of multiple film layers. Every structure has sloped sidewalls with an opening on top and is hollow inside, thus resembling a nano-volcano. By varying the particle diameter-to-wavelength ratio γ, the structures can be designed with and without an inner core, as shown in FIG. 2 a-d and FIG. 2 e-l, respectively. The angle of the sidewall is also dependent on γ and was designed to be 67° to 83°. The results illustrate that the sidewall thickness can be controlled down to approximately 60 nm.

The colloidal light scatterings were described using the FDTD and binary resist models developed, and the nano-volcano dimensions were accurately predicted. FIG. 3 illustrates a quantitative comparison of the patterned and predicted structures using a 1 μm diameter particle and 325 nm wavelength TE-polarized exposure (γ=3.08). The sidewall angles and thicknesses were compared and showed good agreement between the experiment and simulation. Note that the voids in the sidewall of the simulated cross section are enclosed spaces and cannot be dissolved by the developer in the experiments, thus forming a thick solid sidewall. These FDTD and binary resist models were used to predict all simulated nano-volcano structures described herein.

### EXAMPLE 2

Using the hollow shell-like nanostructures fabricated according to embodiments of the method described herein, the trapping capability of the nanostructure was investigated using vacuum techniques. In this approach, the nano-volcano sample was covered by a film of aqueous colloidal solution with 50 nm diameter silica nanoparticles (Polyscience colloidal silica microspheres in 5% aqueous solution) and placed in a vacuum chamber. The vacuum degasses the trapped air bubbles inside the nano-volcano structures, allowing the solution to fill the hollow chambers. The samples were allowed to dry in ambient conditions. The extra particles outside the nanostructures that were not drawn into the nanostructures were removed using ultrasonic agitation, while the particles within the chamber remained trapped.

The particle trapping capability of the fabricated structure is illustrated in FIG. 7 a and b, for example, where the interiors of double-shelled and thick-walled nano-volcanoes after particle loading are shown, respectively. The silica particles can be trapped efficiently within the interior chamber of the nano-volcanoes, while no particles can be observed outside the nano-volcanoes. This trapping mechanism is also uniform, and nano-volcanoes over an area of 5.9 mm² were found loaded.

The double-shelled nano-volcanoes shown in FIG. 7a can collapse under high vacuum because their thin sidewalls (−60 nm) are mechanically unstable. Therefore, thick-walled nano-volcanoes are preferred to withstand high vacuum in this loading mechanism, as indicated in region III of the shape diagram shown in FIG. 4.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A method of making a hollow nanostructure comprising:
positioning a micro- or nano-sized particle over a photoresist;
illuminating the particle, thereby forming a scattering pattern on the photoresist; and
developing the photoresist to reveal the hollow nanostructure defined by the scattering pattern.

2. The method of claim 1, wherein the particle has a diameter of about 50 μm or less.

3. The method of claim 1, wherein the particle has a diameter in the range of about 200 nm to about 2000 nm.

4. The method of claim 3, comprising illuminating the particle with an exposure laser having a wavelength of about 325 nm to about 405 nm.

5. The method of claim 1, wherein the particle has a diameter in the range of about 5 nm to about 50 nm.

6. The method of claim 5, comprising illuminating the particle with an exposure having a wavelength of about 1 nm to about 10 nm.

7. The method of claim 1, wherein the scattering pattern corresponds to a ratio γ of the particle diameter D to the exposure wavelength λ(γ=D/λ).

8. A nanostructure comprising:
a base;
a top with an opening formed thereon; and
a sloped sidewall connecting the base to the top and forming a frusto-conical shape having an angle θ; and
a hollow chamber defined by the base and sidewall and communicating with the top opening.

9. A nanostructure array comprising a plurality of the nanostructures of claim 8.

10. The nanostructure array of claim 9, wherein the plurality of the nanostructures is attached to a backing material.

11. A delivery device comprising a nanostructure array according to claim 9.

12. The nanostructure of claim 8, wherein the angle θ ranges from about 45 to about 90 degrees.

13. A method of forming a nanomaterial delivery device comprising:
providing a hollow nanostructure according to claim 8; and
contacting the hollow nanostructure with a composition comprising a nanomaterial and a carrier so that the nanomaterial enters the hollow chamber.

14. A method of delivering a nanomaterial comprising:
providing a hollow nanostructure according to claim 8;
having the nanomaterial retained within the hollow chamber by a phase change material; and
subjecting the hollow nanostructure to conditions such that the phase change material undergoes a change suitable to allow the nanomaterial to escape from the hollow chamber.
